(19) United States
(12) Patent Application Publication
(10) Pub. No.: US 2004/0077634 A1
(43) Pub. Date: Apr. 22, 2004

(54) Treatment of diseases characterized by excessive or insufficient cell death

(76) Inventors: Ulrik Lademann, Soborg (DK); Marja Jaattela, Kobenhavn (DK); Dan Peters, Malmo (SE); Lars Christian B. Ronn, Vekso (DK)

Correspondence Address: BIRCH STEWART KOLASCH & BIRCH, PO BOX 747, FALLS CHURCH, VA, 22040-0747, US

(21) Appl. No.: 10/467,460

(22) PCT Filed: Feb. 15, 2002
(86) PCT No.: PCT/DK02/00106

(30) Foreign Application Priority Data
Feb. 15, 2001 (DK) PA 2001 00255
Jan. 11, 2002 (DK) PA 2002 00046

(51) Int. Cl. A61K 31/551; A61K 31/4439; A61K 31/4184; A61K 31/195; A61K 31/24

(52) U.S. Cl. 514/220; 514/339; 514/394; 514/535

(57) ABSTRACT

The invention relates to the use of a compound that modulates the association of caspase-9 to Apaf-1 for the treatment of diseases characterized by excessive or insufficient cell death.

### TECHNICAL FIELD

This invention relates to the use of a compound that modulates the association of caspase-9 to Apaf-1 for the treatment of diseases characterized by excessive or insufficient cell death.

### BACKGROUND ART

Apoptosis, or programmed cell death, is a cell suicide mechanism that plays an important role in many physiological processes and its deregulation contributes to many diseases. In the adult animal cells die by apoptosis during tissue turnover and in the end of an immune response. Deregulation of apoptosis is observed in various diseases such as neurodegenerative diseases where cell death is pronounced and cancers where apoptosis is inhibited.

A family of cysteine proteases, the caspases, has been believed to be responsible for the execution of all apoptotic cell death. Caspases can be activated either by the cell surface receptors of the tumour necrosis factor (TNF) family (an extrinsic apoptosis pathway) or by the release of cytochrome c from the mitochondria to the cytosol triggered for example by growth factor deprivation, ischemia or several anticancer drugs (intrinsic apoptosis pathway).

The extrinsic apoptosis pathway is activated by a subfamily of the cell surface receptors, the death receptors, which are a subset of the tumour necrosis factor (TNF) receptor super family. TNF is a multifunctional cytokine that can elicit several biological responses including apoptosis, inflammation and stress response. The numerous biological effects of TNF are signalled via two distinct cell surface receptors, THF-R1 and TNF-R2, the former being the major signalling receptor in most cells. TNF-R1 trimerizes upon TNF binding, which leads to the subsequent recruitment and binding of other intracellular death domain containing proteins through death domain interaction. Caspase-8 is activated when recruited to the receptor complex and can in turn activate effector caspases (i.a. caspase-3) followed by apoptosis.

The mitochondria are the centre of the intrinsic apoptosis pathway. In response to various stimuli, the mitochondria release cytochrome c to the cytosol. In the cytosol, cytochrome c induces an ATP/dATP dependent formation of a protein complex named the “apoptosome”. Apoptosome consists of Apaf-1 (Apoptotic protease activating factor-1), caspase-9, and cytochrome c. Within this complex caspase-9 is activated to cleave and activate the effector caspases, caspase-3 and caspase-7.

Consequently, the modulation of caspase activity by therapeutic intervention might prove useful in the treatment of diseases characterized by excessive or insufficient cell death.

### SUMMARY OF THE INVENTION

According to the invention it has now been found that a compound that modulates the association of caspase-9 to Apaf-1 can be used for the treatment of a disease being characterized by excessive or insufficient cell death.

Accordingly, in its first aspect, the invention relates to the use of a compound that modulates the association of caspase-9 to Apaf-1 or a pharmaceutically acceptable salt thereof for the manufacture of a medicament for the treatment, prevention or alleviation of a disease in a subject, said disease being characterized by excessive or insufficient cell death.

In another aspect, the invention relates to novel compounds being able to inhibit the association of caspase-9 to Apaf-1.

The compounds can also be utilized in vitro as unique research tools for understanding, inter alia, how apoptosis is regulated as the cellular level.

Other objects of the invention will be apparent to the person skilled in the art from the following detailed description and examples.

### DETAILED DISCLOSURE OF THE INVENTION

In its first aspect, the invention provides the use of a compound that modulates the association of caspase-9 to Apaf-1 or a pharmaceutically acceptable salt thereof for the manufacture of a medicament for the treatment, prevention or alleviation of a disease in a subject, said disease being characterized by excessive or insufficient cell death.

In a second aspect, the invention provides the use of a compound that inhibits the association of caspase-9 to Apaf-1 or a pharmaceutically acceptable salt thereof for the manufacture of a medicament for the treatment, prevention or alleviation of a disease in a subject, said disease being characterized by excessive cell death.

In a third aspect, the invention provides a method of treatment, prevention r alleviation of a disease in a subject, said disease being characterized by excessive r insufficient cell death, which method comprises administering to said subject a therapeutically effective amount of a compound that modulates the association of caspase-9 to Apaf-1 or a pharmaceutically acceptable amount thereof.

In a further aspect, the invention provides a method of treatment, prevention or alleviation of a disease in a subject, said disease being characterized by excessive cell death, which method comprises administering to said subject a therapeutically effective amount of a compound that inhibits the association of caspase-9 to Apaf-1 or a pharmaceutically acceptable amount thereof.

In a still further aspect, the invention provides a novel compound being

[N-(2-Hydroxy-5-methoxycarbonyl-4-nitrophenyl)-N′-(3-trifluoromethylphenyl)]urea;

N-(2-Hydroxy-5-chloropheneth-2-yl)-3-trifluoromethylaniline;

[N-(2,4-Dihydroxyphenyl)-N′-(3-trifluoromethylphenyl)]urea;

1,2,4-Oxadiazolo[3,4-d]benz[b]-1,4-diazin-1-one;

1-[3-(2-Amino-pyrid-5-yl)-phen-1-yl]-5-trifluoromethyl-benzimidazole;

5-Formamidyl-1-(3-biphenyl)-benzimidazole;

1-(3-Aminophenyl-3-phen-1-yl)-2-trifluoromethyl-benzimidazole;

1-(3-Biphenyl)-5-methoxy-benzimidazole;

[N-(2-Hydroxy-5-methoxy-4-nitrophenyl)-N′-(3-trifluoromethylphenyl)]urea;

γ-(5-Chloro-2-hydroxyphenyl)-(3-trifluoromethylacetanilide),

N-(3-Trifluoromethyl-pheneth-2-yl)-5-chloro-2-hydroxy-aniline;

[N-(5-Carboxy-2-hydroxy-4-nitrophenyl)-N-(3-trifluoromethylphenyl)]urea;

(1R,2S,3S)-2-(3-Phenyl-1,2,4-oxadiazol-5-yl)-3-(2-naphthyl)-tropane;

[N-(2-Hydroxy-5-trifluoromethylphenyl)-N′-(3-trifluoromethylphenyl)]urea;

N,N ′-Bis-(2-hydroxy-5-trifluoromethylphenyl)-urea;

7,8-Dichloro-1,2,4-oxadiazolo[3,4-d]benz[b]-1,4-diazin-1-one;

7-Nitro-1,2,4-oxadiazolo[3,4-d]benz[b]-1,4-diazin-1-one;

5-Amino-1-(3-biphenyl)-benzimidazole;

1-(3-Biphenyl)-benzimidazole;

(1R,2S,3S)-2-[3-(2-Thienyl)-1,2,4-oxadiazol-5-yl]-3-(2-naphthyl)-tropane;

(1R,2S,3S)-N-Normethyl-2-(3-phenyl-1,2,4-oxadiazol-5-yl)-3-(3,4-dichlorophenyl)-tropane;

(1R,2S,3S)-N-Normethyl-2-(3-phenyl-1,2,4-oxadiazol-5-yl)-3-(4-chlorophenyl)-tropane;

or pharmaceutically acceptable salts thereof.

In a further aspect, the invention relates to a pharmaceutical composition containing a therapeutically effective amount of a novel compound as described above, or a pharmaceutically acceptable addition salt thereof, together with at least one pharmaceutically acceptable carrier, excipient or diluent.

In one embodiment, the compound that inhibits the association of caspase9 to Apaf-1 is a nonpeptide compound.

In a second embodiment, the compound that inhibits the association of caspase-9 to Apaf-1 is a compound of general formula I

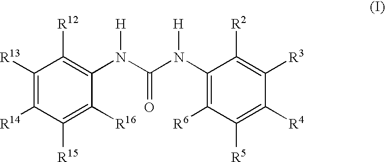

or a pharmaceutically acceptable salt thereof;

wherein

R² represents —OH or —COOH;

R³, R⁴, R⁵, and R⁶ independently of each represent hydrogen, halogen, hydroxy, amino, cyano, nitro, trifluoromethyl, —CO₂R¹, or —COR¹;

wherein R¹ is hydrogen or alkyl;

one of R¹², R¹³, R¹⁴, R¹⁵, and R¹⁵ represents trifluoromethyl; and

the other four of R¹², R¹³, R¹⁴, R¹⁵, and R¹⁵ represent hydrogen.

In a special embodiment of the compound of general formula 1, R² represents —OH. In a further embodiment, R² represents —COOH. In a still further embodiment, R⁴ represents hydroxy. In a further embodiment, R⁴ represents nitro. In a still further embodiment R⁵ represents —COR¹, wherein R¹ is alkyl, such as methyl. In a further embodiment R⁵ represents halogen, such as chloride.

In a further embodiment, the compound that inhibits the association of caspase-9 to Apaf-1 is selected from:

[N-(2-Hydroxy-5-methoxycarbonyl-4-nitrophenyl)-N′-(3-trifluoromethylphenyl)]urea;

N-(2-Hydroxy-5-chloropheneth-2-yl)-3-trifluoromethylaniline;

[N-(2,4-Dihydroxyphenyl)-N′-(3-trifluoromethylphenyl)]urea;

1,2,4-Oxadiazolo[3,4-d]benz[b]-1,4-diazin-1-one;

1-[3-(2-Amino-pyrid-5-yl)-phen-1-yl]-5-trifluoromethyl-benzimidazole;

5-Formamidyl-1-(3-biphenyl)-benzimidazole;

1-(3-Aminophenyl-3-phen-1-yl)-2-trifluoromethyl-benzimidazole;

1-(3-Biphenyl)-5-methoxy-benzimidazole;

[N-(2-Hydroxy-5-methoxy-4-nitrophenyl)-N′-(3-trifluoromethylphenyl)]urea;

γ-(5-Chloro-2-hydroxyphenyl)-(3-trifluoromethylacetanilide),

N-(3-Trifluoromethyl-pheneth-2-yl)-5-chloro-2-hydroxy-aniline;

[N-(5-Carboxy-2-hydroxy-4-nitrophenyl)-N′-(3-trifluoromethylphenyl)]urea;

(1R,2S,3S)-2-(3-Phenyl-1,2,4-oxadiazol-5-yl)-3-(2-naphthyl)-tropane;

[N-(2-Hydroxy-5-trifluoromethylphenyl)-N′-(3-trifluoromethylphenyl)]urea;

N,N′-Bis-(2-hydroxy-5-trifluoromethylphenyl)-urea;

7,8-Dichloro-1,2,4-oxadiazolo[3,4-d]benz[b]-1,4-diazin-1-one;

7-Nitro-1,2,4-oxadiazolo[3,4-d]benz[b]-1,4-diazin-1-one;

5-Amino-1-(3-biphenyl)-benzimidazole;

1-(3-Biphenyl)-benzimidazole;

[N-(2-Carboxy-5-chlorophenyl)-N′-(3-trifluoromethylphenyl)]urea;

(1S,3S,4S,5S,8R)-3-(4-Chlorophenyl)-7-azatricyclo[5.3.0.0]decan-5-oxime;

(1R,2S,3S)-2-[3-(2-Thienyl)-1,2,4-oxadiazol-5-yl]-3-(2-naphthyl)-tropane;

(1R,2S,3S)-N-Normethyl-2-(3-phenyl-1,2,4-oxadiazol-5-yl)-3-(3,4-dichlorophenyl)-tropane;

[N-(2-Carboxy-5-chlorophenyl)-N′-(4-trifluoromethylphenyl)]urea;

[N-(2-Carboxy-5-chlorophenyl)-N′-(3-nitrophenyl)]urea;

(1S,3S,4S,5S,8R)-3-(3,4-Dichlorophenyl)-7-azatricyclo[5.3.0.0]decan-5-oxime;

(1R,2S,3S)-N-Normethyl-2-(3-phenyl-1,2,4-oxadiazol-5-yl)-3-(4-chlorophenyl)-tropane;

or pharmaceutically acceptable salts thereof.

In a still further embodiment, the disease being characterized by excessive cell death is a neurodegenerative disorder or ischemia, such as cerebral ischemia. In a special embodiment, the disease being characterized by excessive cell death is a neurodegenerative disorder. In a further special embodiment, the disease being characterized by excessive cell death is ischemia, such as cerebral ischemia.

The subject to be treated according to this invention is a living body, preferably a mammal, most preferably a human, in need for such treatment.

Any possible combination of two or more of the embodiments described described herein is comprised within the scope of the present application.

Compounds that modulates the association of caspase-9 to Apa1-1

The potential of a given substance to act as a compound that modulates the association of caspase-9 to Apaf-1 may be determined using standard in vitro assays, such as those described in “Test methods”.

In one embodiment, the compound that inhibits the association of caspase-9 to Apaf-1 shows more 50% inhibition, preferably more than 60% inhibition, more preferably more than 70% inhibition, and even more preferably more than 80% inhibition, when tested in the DEVDase assay (method 1).

Novel Compounds

The novel compounds of the invention may be prepared by conventional methods for chemical synthesis. All N,N′-diarylureas were prepared by mixing the corresponding arylurea and arylisocyanate in toluene.

The end products of the reactions described herein may be isolated by conventional techniques, e.g. by extraction, crystallisation, distillation, chromatography, etc.

The following novel compounds were prepared using conventional methods, such as those described in various published NeuroSearch patent applications:

[N-(2-Hydroxy-5-methoxycarbonyl-4-nitrophenyl)-N′-(3-trifluoromethylphenyl)]urea, mp 101-102° C.

(1R,2S,3S)-2-[3-(2-Thienyl)-1,2,4-oxadiazol-5-yl]-3-(2-naphthyl)-tropane hydrochloric acid salt, mp 149-150° C.

N-(2-Hydroxy-5-chloropheneth-2-yl)-3-trifluoromethylaniline hydrochloric acid salt, mp 182° C.

(1R,2S,3S)-N-Normethyl-2-(3-phenyl-1,2,4-oxadiazol-5-yl)-3-(3,4-dichlorophenyl)-tropane hydrochloric acid salt, mp 150° C.

[N-(2,4-Dihydroxyphenyl)-N′-(3-trifluoromethylphenyl)]urea, mp 179-180° C. 1,2,4-Oxadiazolo[3,4-d]benz[b]-1,4-diazin-1-one, mp 153-154° C.

1-[3-(2-Amino-pyrid-5-yl)-phen-1-yl]-5-trifluoromethyl-benzimidazole hydrochloric acid salt, mp 292-294° C.

5-Formamidyl-1-(3-biphenyl)-benzimidazole, mp 169-170° C.

1-(3-Aminophenyl-3-phen-1-yl)-2-trifluoromethyl-benzimidazole, mp 48-50° C.

1-(3-Biphenyl)-5-methoxy-benzimidazole, mp 111-112° C.

[N-(2-Hydroxy-5-methoxy-4-nitrophenyl)-N′-(3-trifluoromethylphenyl)]urea, mp 220-222° C.

γ-(5-Chloro-2-hydroxyphenyl)-(3-trifluoromethylacetanilide), mp 148-150° C.

(1R,2S,3S)-N-Normethyl-2-(3-phenyl-1,2,4-oxadiazol-5-yl)-3-(4-chlorophenyl)-tropane hydrochloric acid salt, mp 185-187° C.

N-(3-Trifluoromethyl-pheneth-2-yl)-5-chloro-2-hydroxy-aniline, mp 92-95° C.

[N-(5-Carboxy-2-hydroxy-4-nitrophenyl)-N′-(3-trifluoromethylphenyl)]urea, mp 201-203° C.

(1R,2S,3S)-2-(3-Phenyl-1,2,4-oxadiazol-5-yl)-3-(2-naphthyl)-tropane hydrochloric acid salt, amorphous material.

[N-(2-Hydroxy-5-trifluoromethylphenyl)-N′-(3-trifluoromethylphenyl)]urea, mp 160-162° C.

N,N′Bis-(2-hydroxy-5-trifluoromethylphenyl)-urea, mp 175-176° C.

7,8-Dichloro-1,2,4-oxadiazolo[3,4-d]benz[b]-1,4-diazin-1-one, mp 164-167° C.

7-Nitro-1,2,4-oxadiazolo[3,4-d]benz[b]-1,4-diazin-1-one, mp 218° C.

5-Amino-1-(3-biphenyl)-benzimidazole, hydrochloric acid salt, mp 147-149° C.

1-(3-Biphenyl)-benzimidazole, oil.

Pharmaceutically Acceptable Salts

The chemical compound of the invention may be provided in any form suitable for the intended administration. Suitable forms include pharmaceutically (i.e. physiologically) acceptable salts, and pre- or prodrug forms of the chemical compound of the invention.

Examples of pharmaceutically acceptable addition salts include, without limitation, the non-toxic inorganic and organic acid addition salts such as the hydrochloride derived from hydrochloric acid, the hydrobromide derived from hydrobromic acid, the nitrate derived from nitric acid, the perchlorate derived from perchloric acid, the phosphate derived from phosphoric acid, the sulphate derived from sulphuric acid, the formate derived from formic acid, the acetate derived from acetic acid, the aconate derived from aconitic acid, the ascorbate derived from ascorbic acid, the benzenesulphonate derived from benzensulphonic acid, the benzoate derived from benzoic acid, the cinnamate derived from cinnamic acid, the citrate derived from citric acid, the embonate derived from embonic acid, the enantate derived from enanthic acid, the fumarate derived from fumaric acid, the glutamate derived from glutamic acid, the glycolate derived from glycolic acid, the lactate derived from lactic acid, the maleate derived from maleic acid, the malonate derived from malonic acid, the mandelate derived from mandelic acid, the methanesulphonate derived from methane sulphonic acid, the naphthalene-2-sulphonate derived from naphtalene-2-sulphonic acid, the phthalate derived from phthalic acid, the salicylate derived from salicylic acid, the sorbate derived from sorbic acid, the stearate derived from stearic acid, the succinate derived from succinic acid, the tartrate derived from tartaric acid, the toluene-p-sulphonate derived from p-toluene sulphonic acid, and the like. Such salts may be formed by procedures well known and described in the art.

Other acids such as oxalic acid, which may not be considered pharmaceutically acceptable, may be useful in the preparation of salts useful as intermediates in obtaining a chemical compound of the invention and its pharmaceutically acceptable acid addition salt.

Metal salts of a chemical compound of the invention includes alkali metal salts, such as the sodium salt of a chemical compound of the invention containing a carboxy group.

In the context of this invention the “onium salts” of N-containing compounds are also contemplated as pharmaceutically acceptable salts. Preferred “onium salts” include the alkyl-onium salts, the cycloalkyl-onium salts, and the cycloalkylalkyl-onium salts.

Th chemical compound of the invention may be provided in dissoluble or indissoluble forms together with a pharmaceutically acceptable solvents such as water, ethanol, and the like. Dissoluble forms may also include hydrated forms such as the monohydrate, the dihydrate, the hemihydrate, the trihydrate, the tetrahydrate, and the like. In general, the dissoluble forms are considered equivalent to indissoluble forms for the purposes of this invention.

Prodrugs

The chemical compound of the invention may be administered as such or in the form of a suitable prodrug.

The term “prodrug” denotes a compound, which is a drug precursor and which, following administration and absorption, release the drug in vivo via some metabolic process.

Particularly favoured prodrugs are those that increase the bioavailability of the compounds of the invention (e.g. by allowing an orally administrered compound to be more readily absorbed into the blood) or which enhance delivery of the parent compound to a specific biological compartment (e.g. the brain or lymphatic system).

Thus examples of suitable prodrugs of the substances according to the invention include compounds modified at one or more reactive or derivatizable groups of the parent compound. Of particular interest are compounds modified at a carboxyl group, a hydroxyl group, or an amino group. Examples of suitable derivatives are esters or amides.

Steric Isomers

The chemical compounds of the present invention may exist in (+) and (−) forms as well as in racemic forms. The racemates of these isomers and the individual isomers themselves are within the scope of the present invention.

Racemic forms can be resolved into the optical antipodes by known methods and techniques. One way of separating the diastereomeric salts is by use of an optically active acid, and liberating the optically active amine compound by treatment with a base. Another method for resolving racemates into the optical antipodes is based upon chromatography on an optical active matrix. Racemic compounds of the present invention can thus be resolved into their optical antipodes, e.g., by fractional crystallisation of d- or l-(tartrates, mandelates, or camphorsulphonate) salts for example.

The chemical compounds of the present invention may also be resolved by the formation of diastereomeric amides by reaction of the chemical compounds of the present invention with an optically active activated carboxylic acid such as that derived from (+) or (−) phenylalanine, (+) or (−) phenylglycine, (+) or (−) camphanic acid or by the formation of diastereomeric carbamates by reaction of the chemical compound of the present invention with an optically active chloroformate or the like.

Additional methods for the resolving the optical isomers are known in the art. Such methods include those described by Jaques J, Collet A, & Wilen S in “Enantiomers, Racemates, and Resolutions”, John Wiley and Sons, New York (1981).

Optical active compounds can also be prepared from optical active starting materials.

Definition of Substituents

In the context of this invention halogen represents a fluorine, a chlorine, a bromine or an iodine atom.

Alkyl means a straight chain or branched chain of one to six carbon atoms, including but not limited to, methyl, ethyl, propyl, isopropyl, butyl, isobutyl, t-butyl, pentyl, and hexyl; methyl, ethyl, propyl and isopropyl are preferred groups.

Pharmaceutical Compositions

While a chemical compound of the invention for use in therapy may be administered in the form of the raw chemical compound, it is preferred to introduce the active ingredient, optionally in the form of a physiologically acceptable salt, in a pharmaceutical composition together with one or more adjuvants, excipients, carriers, buffers, diluents, and/or other customary pharmaceutical auxiliaries.

In a preferred embodiment, the invention provides pharmaceutical compositions comprising the chemical compound of the invention, or a pharmaceutically acceptable salt or derivative thereof, together with one or more pharmaceutically acceptable carriers therefor, and, optionally, other therapeutic and/or prophylactic ingredients. The carrier(s) must be “acceptable” in the sense of being compatible with the other ingredients of the formulation and not harmful to the recipient thereof.

Pharmaceutical compositions of the invention may be those suitable for oral, rectal, bronchial, nasal, topical (including buccal and sub-lingual), transdermal, vaginal or parenteral (including cutaneous, subcutaneous, intramuscular, intraperitoneal, intravenous, intraarterial, intracerebral, intraocular injection or infusion) administration, or those in a form suitable for administration by inhalation or insufflation, including powders and liquid aerosol administration, or by sustained release systems. Suitable examples of sustained release systems include semipermeable matrices of solid hydrophobic polymers containing the compound of the invention, which matrices may be in form of shaped articles, e.g. films or microcapsules.

The chemical compound of the invention, together with a conventional adjuvant, carrier, or diluent, may thus be placed into the form of pharmaceutical compositions and unit dosages thereof. Such forms include solids, and in particular tablets, filled capsules, powder and pellet forms, and liquids, in particular aqueous or non-aqueous solutions, suspensions, emulsions, elixirs, and capsules filled with the same, all for oral use, suppositories for rectal administration, and sterile injectable solutions for parenteral use. Such pharmaceutical compositions and unit dosage forms thereof may comprise conventional ingredients in conventional proportions, with or without additional active compounds or principles, and such unit dosage forms may contain any suitable effective amount of the active ingredient commensurate with the intended daily dosage range to be employed.

The chemical compound of the present invention can be administered in a wide variety of oral and parenteral dosage forms. It will be obvious to those skilled in the art that the following dosage forms may comprise, as the active component, either a chemical compound of the invention or a pharmaceutically acceptable salt of a chemical compound of the invention.

For preparing pharmaceutical compositions from a chemical compound of the present invention, pharmaceutically acceptable carriers can be either solid or liquid. Solid form preparations include powders, tablets, pills, capsules, cachets, suppositories, and dispersible granules. A solid carrier can be one or more substances which may also act as diluents, flavouring agents, solubilizers, lubricants, suspending agents, binders, preservatives, tablet disintegrating agents, or an encapsulating material.

In powders, the carrier is a finely divided solid, which is in a mixture with the finely divided active component.

In tablets, the active component is mixed with the carrier having the necessary binding capacity in suitable proportions and compacted in the shape and size desired.

The powders and tablets preferably contain from five or ten to about seventy percent of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as carrier providing a capsule in which the active component, with or without carriers, is surrounded by a carrier, which is thus in association with it. Similarly, cachets and lozenges are included. Tablets, powders, capsules, pills, cachets, and lozenges can be used as solid forms suitable for oral administration.

For preparing suppositories, a low melting wax, such as a mixture of fatty acid glyceride or cocoa butter, is first melted and the active component is dispersed homogeneously therein, as by stirring. The molten homogenous mixture is then poured into convenient sized moulds, allowed to cool, and thereby to solidify.

Compositions suitable for vaginal administration may be presented as pessaries, tampons, creams, gels, pastes, foams or sprays containing in addition to the active ingredient such carriers as are known in the art to be appropriate.

Liquid preparations include solutions, suspensions, and emulsions, for example, water or water-propylene glycol solutions. For example, parenteral injection liquid preparations can be formulated as solutions in aqueous polyethylene glycol solution.

The chemical compound according to the present invention may thus be formulated for parenteral administration (e.g. by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulation agents such as suspending, stabilising and/or dispersing agents. Alternatively, the active ingredient may be in powder form, obtained by aseptic isolation of sterile solid or by lyophilization from solution, for constitution with a suitable vehicle, e.g. sterile, pyrogen-free water, before use.

Aqueous solutions suitable for oral use can be prepared by dissolving the active component in water and adding suitable colorants, flavours, stabilising and thickening agents, as desired.

Aqueous suspensions suitable for oral use can be made by dispersing the finely divided active component in water with viscous material, such as natural or synthetic gums, resins, methylcellulose, sodium carboxymethylcellulose, or other well known suspending agents.

Also included are solid form preparations, intended for conversion shortly before use to liquid form preparations for oral administration. Such liquid forms include solutions, suspensions, and emulsions. In addition to the active component such preparations may comprise colorants, flavours, stabilisers, buffers, artificial and natural sweeteners, dispersants, thickeners, solubilizing agents, and the like.

For topical administration to the epidermis the chemical compound of the invention may be formulated as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also contain one or more emulsifying agents, stabilising agents, dispersing agents, suspending agents, thickening agents, or colouring agents.

Compositions suitable for topical administration in the mouth include lozenges comprising the active agent in a flavoured base, usually sucrose and acacia or tragacanth; pastilles comprising the active ingredient in an inert base such as gelatin and glycerine or sucros and acacia; and mouthwashes comprising th active ingredient in a suitable liquid carrier.

Solutions or suspensions are applied directly to the nasal cavity by conventional means, for example with a dropper, pipette or spray. The compositions may be provided in single or multi-dose form.

Administration to the respiratory tract may also be achieved by means of an aerosol formulation in which the active ingredient is provided in a pressurised pack with a suitable propellant such as a chlorofluorocarbon (CFC) for example dichlorodifluoromethane, trichlorofluoromethane, or dichlorotetrafluoroethane, carbon dioxide, or other suitable gas. The aerosol may conveniently also contain a surfactant such as lecithin. The dose of drug may be controlled by provision of a metered valve.

Alternatively the active ingredients may be provided in the form of a dry powder, for example a powder mix of the compound in a suitable powder base such as lactose, starch, starch derivatives such as hydroxypropylmethyl cellulose and polyvinylpyrrolidone (PVP). Conveniently the powder carrier will form a gel in the nasal cavity. The powder composition may be presented in unit dose form for example in capsules or cartridges of, e.g., gelatin, or blister packs from which the powder may be administered by means of an inhaler.

In compositions intended for administration to the respiratory tract, including intranasal compositions, the compound will generally have a small particle size for example of the order of 5 microns or less. Such a particle size may be obtained by means known in the art, for example by micronization.

When desired, compositions adapted to give sustained release of the active ingredient may be employed.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packaged tablets, capsules, and powders in vials or ampoules. Also, the unit dosage form can be a capsule, tablet, cachet, or lozenge itself, or it can be the appropriate number of any of these in packaged form.

Tablets or capsules for oral administration and liquids for intravenous administration and continuous infusion are preferred compositions.

Further details on techniques for formulation and administration may be found in the latest edition of Remington's Pharmaceutical Sciences (Maack Publishing Co., Easton, Pa.).

A therapeutically effective dose refers to that amount of active ingredient, which ameliorates the symptoms or condition. Therapeutic efficacy and toxicity, e.g. ED₅₀ and LD₅₀, may be determined by standard pharmacological procedures in cell cultures or experimental animals. The dose ratio between therapeutic and toxic effects is the therapeutic index and may be expressed by the ratio LD₅₀/LD₅₀. Pharmaceutical compositions exhibiting large therapeutic indexes are preferred.

The dose administered must of course be carefully adjusted to the age, weight and condition of the individual being treated, as well as the route of administration, dosage form and regimen, and the result desired, and the exact dosage should of course be determined by the practitioner.

The actual dosage depend on the nature and severity of the disease being treated and the route of administration, and is within the discretion of the physician, and may be varied by titration of the dosage to the particular circumstances of this invention to produce the desired therapeutic effect. However, it is presently contemplated that pharmaceutical compositions containing of from about 0.01 to about 500 mg of active ingredient per individual dose, preferably of from about 0.1 to about 100 mg, most preferred of from about 1 to about 10 mg, are suitable for therapeutic treatments.

The active ingredient may be administered in one or several doses per day. A satisfactory result can, in certain instances, be obtained at a dosage as low as 0.01 μg/kg i.v. and 0.1 μg/kg p.o. The upper limit of the dosage range is presently considered to be about 10 mg/kg i.v. and 100 mg/kg p.o. Preferred ranges are from about 0.1 μg/kg to about 10 mg/kg/day i.v., and from about 1 μg/kg to about 100 mg/kg/day p.o.

The invention is further illustrated with reference to the following test methods and examples, which are not intended to be in any way limiting to the scope of the invention as claimed.

### Test Methods

Method 1

Inhibition of DEVDase

In this experiment, the effect of a compound with neurotrophic activity (below: the compound) on the inhibition of DEVDase (caspase-3-like protease) is assessed.

Preparation of Cell Extract

Hela cells are grown in large petri dishes until subconfluency. Cells are then harvested and washed in PBS.

The cell pellet is resuspended in equal volumes of ice-cold isotonic lysisbuffer (SCA; 20 mM Hepes-KOH pH 7.5, 10 mM KCl, 1.5 mM MgCl₂, 1 mM EDTA, 1 mM EGTA, 250 mM sucrose, 1 mM DTT, 10 μg/ml aprotinin, 1 μg/ml leupeptin, 1 μg/ml pepstatin A, 100 μg/ml peta-block), incubated on ice for 30 min and lysed by approx. 30 strokes of a dounce homogenizer.

The lysate is then centrifuged at 750 g for 10 min, and the supernatant thereof at 10,000 g for 10 min and at 20,000 g for 30 min. The clarified supernatant is removed carefully and stored in aliquots at −80° C.

In Vitro Activation of DEVDases

Activation of endogene caspases is induced by addition of 1 mM dATP and 1 μM horse heart cytochrome c to cytosolic extract (protein concentration; 5-10 mg/ml) in the presence of DEVD-AFC and the compound (final concentration 100 μM). Activated DEVD'ases (AFC release) is measured by fluorometry in 96-well plates at 37° C. After end measurements 2× LSB-Mechaptoethanol are added to the samples for assessment of caspase processing by immunoblot analysis.

Percent inhibition is calculated as follows:

$$[ 1 - \frac{Experimental treatment - minimal activity}{maximum activity - minimal activity} ] \times 100 \%$$

Maximal activity was defined as DEVDase activity (Vₘₐₓ) induced by cytochrome c/dATP and minimal activity was defined as DEVDase activity (Vₘᵢₙ) induced by cytochrome c/dATP stimulation+0.1 μM DEVD-CHO or 1 μM zVAD-fmk (two known caspase inhibitors).

### EXAMPLES

The invention is further illustrated with reference to the following examples, which are not intended to be in any way limiting to the scope of the invention as claimed.

### Example 1

Compounds of the invention were tested for their effect on inhibition of DEVDase (test method 1). Test results for a number of compounds are listed below:

| Com- | | % |
|---|---|---|
| pound | Compound | Inhibition |
| a | [N-(2-Hydroxy-5-methoxycarbonyl-4-nitrophenyl)- | 80 |
| | N′-(3-trifluoromethylphenyl)]urea | |
| b | N-(2-Hydroxy-5-chloropheneth-2-yl)-3- | 75 |
| | trifluoromethylaniline hydrochloric acid salt | |
| c | (1R,2S,3S)-2-[3-(2-Thienyl)-1,2,4-oxadiazol-5-yl]- | 60 |
| | 3-(2-naphthyl)-tropane hydrochloric acid salt, | |
| d | 1-(3-Biphenyl)-benzimidazole | 58 |
| e | 7-Nitro-1,2,4-oxadiazolo[3,4-d]benz[b]-1,4-diazin- | 63 |
| | 1-one | |
| f | [N-(2-Carboxy-5-chlorophenyl)-N′-(3- | 94 |
| | trifluoromethylphenyl)]urea | |
| g | [N-(2,4-Dihydroxyphenyl)-N′-(3- | 74 |
| | trifluoromethylphenyl)]urea | |

### Example 2

The inhibition of the DEVDase activity in the in vitro caspase activation system could be due to a direct inhibition of caspase-9, -3, or -7 activities, or to the inhibition of the apoptosome formation. To test if compounds a, f, and g were specific caspase inhibitors, recombinant caspase-3 and caspase-9 were incubated with the three compounds and the enzymatic activities of caspases were monitored by analysing the cleavage of the caspase-3 substrate DEVD-AFC or the caspase-9 substrate LEHD-AFC, respectively. All three compounds at concentrations ranging from 25 to 100 μM failed to inhibit the activities of recombinant caspase-9 and caspase-3 significantly, whereas 0.1 μM DEVD-CHO or 1 μM zVAD-fmk (two known caspase inhibitors) conferred almost total inhibition

Next, the effect of the compounds on the processing of caspases and caspase substrates in the in vitro model system was analysed by Western blotting. Caspase-9, caspase-3 and caspase-7 were processed into p35, p17, and p20 active fragments, respectively, in response to Cc/dATP. Accordingly, Cc/dATP induced a threefold induction in DEVDase activity and cleavage of the caspase-3 substrates DFF45/ICAD and PARP. When cell extracts were stimulated with Cc/dATP in the presence of compound f, a dose-dependent inhibition of caspase-9, -3 and -7 processing as well as DFF45/ICAD and PARP cleavage was observed.

This was accompanied by a dose-dependent inhibition of the DEVDase activity. The processing of caspase-9 was almost completely inhibited by 100 μM compound f but even at 10 μM, slightly more procaspase-9 remained unprocessed when compared with the vehicle control. A dose-dependent inhibition was also observed for compounds a and g.

The results suggest that compound f inhibits the Cc/dATP-induced caspase activation upstream of the caspase-9 activation.

### Example 3

Next, it was tested whether compound f interferes with the formation of apoptosome complex in response to Cc/dATP. Cell extracts were incubated with Cc/dATP in the presence or absence of 100 μM compound f or 1 μM zVAD-fmk for 1 hour prior to the immunoprecipitation (IP) of caspase-9. Caspase-9 antibody precipitated practically all caspase-9 present in the cell extract and as expected, Apaf-1 was efficiently co-immunoprecipitated with caspase-9 even in the presence of zVAD-fmk. Compound f had no effect on the ability of the caspase-9 antibody to precipitate caspase-9, but it significantly inhibited the co-immunoprecipitation of Apaf-1 and caspase-9. Hsp90, which has been proposed to associate with Apaf-1 and prevent oligomerization and caspase-9 recruitment, was not co-immunoprecipitated with caspase-9.

### Example 4

The ability of compound f to inhibit cell death was examined in MCF-neo and MCF-casp3 cells. TNF-induced death of both MCF-neo and MCF-casp3 cells was blocked completely by the treatment of the cells with 50 μM zVAD-fmk. DEVD-CHO, an inhibitor of caspase-3 like proteases, blocked cell death induced by 1 ng/ml TNF and conferred significant protection against 5 ng/ml TNF at the concentration of 200 μM. Cell death induced in MCF-neo and MCF-casp3 cells by 1 ng/ml TNF was completely blocked by 50 μM of compound f, and the protection was still significant following treatment with 5 ng/ml TNF. When cells were treated with 10 ng/ml TNF, no inhibition by compound f was observed. To analyse the mechanism of the action of compound f in alive cells, MCF-casp3 cells were treated with TNF (5 ng/ml) in the presence of 50 μM compound f and the activity of caspase-3-like proteases was measured by a DEVDase enzyme assay. Compound f significantly reduced the TNF-induced DEVDase activity.

Finally, it was studied whether compound f treatment inhibits TNF-induced caspase-independent cell death of the WEHI-S fibrosarcoma cells (as described in Foghsgaard et al, the Journal of Cell Biology, Volume 153, Number 5, May 28, 2001, pp 999-1009). No inhibition of the TNF-induced cell death was observed when WEHI-S cells were incubated with compound f (50-10 μM), suggesting that compound f does not inhibit caspase-independent cell death.

1. The use of a compound that modulates the association of caspase-9 to Apaf-1 or a pharmaceutically acceptable salt thereof
for the manufacture of a medicament for the treatment, prevention or alleviation of a disease in a subject, said disease being characterized by excessive or insufficient cell death.

2. The use of a compound that inhibits the association of caspase-9 to Apaf-1 or a pharmaceutically acceptable salt thereof
for the manufacture of a medicament for the treatment, prevention or alleviation of a disease in a subject, said disease being characterized by excessive cell death.

3. The use according to claim 2, wherein the compound that inhibits the association of caspase-9 to Apaf-1 is a compound of general formula I 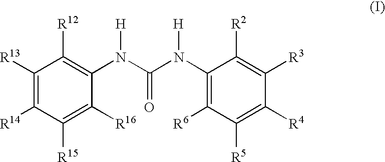
or a pharmaceutically acceptable salt thereof;
wherein
R² represents —OH or —COOH;
R³, R⁴, R⁵, and R⁶ independently of each represent hydrogen, halogen, hydroxy, amino, cyano, nitro, trifluoromethyl, —CO₂R¹, or —COR¹;
wherein R¹ is hydrogen or alkyl;
one of R¹², R¹³, R¹⁴, R¹⁵, and R¹⁵ represents trifluoromethyl; and
the other four of R¹², R¹³, R¹⁴, R¹⁵, and R¹⁵ represent hydrogen.

4. The use according to claim 2, wherein the compound that inhibits the association of caspase-9 to Apaf-1 is selected from:
[N-(2-Hydroxy-5-methoxycarbonyl-4-nitrophenyl)-N′-(3-trifluoromethylphenyl)]urea;
N-(42-Hydroxy-5-chloropheneth-2-yl)-3-trifluoromethylaniline;
[N-(2,4-Dihydroxyphenyl)-N′-(3-trifluoromethylphenyl)]urea;
1,2,4-Oxadiazolo[3,4-d]benz[b]-1,4-diazin-1-one;
1-[3-(2-Amino-pyrid-5-yl)-phen-1-yl]-5-trifluoromethyl-benzimidazole;
5-Formamidyl-1-(3-biphenyl)-benzimidazole;
1-(3-Aminophenyl-3-phen-1-yl)-2-trifluoromethyl-benzimidazole;
1-(3-Biphenyl)-5-methoxy-benzimidazole;
[N-(2-Hydroxy-5-methoxy-4-nitrophenyl)-N′-(3-trifluoromethylphenyl)]urea;
γ-(5-Chloro-2-hydroxyphenyl)-(3-trifluoromethylacetanilide),
N-(3-Trifluoromethyl-pheneth-2-yl)-5-chloro-2-hydroxy-aniline;
[N-(5-Carboxy-2-hydroxy-4-nitrophenyl)-N′-(3-trifluoromethylphenyl)]urea;
(1R,2S,3S)-2-(3-Phenyl-1,2,4-oxadiazol-5-yl)-3-(2-naphthyl)-tropane;
[N-(2-Hydroxy-5-trifluoromethylphenyl)-N′-(3-trifluoromethylphenyl)]urea;
N,N′-Bis-(2-hydroxy-5-trifluoromethylphenyl)-urea;
7,8-Dichloro-1,2,4-oxadiazolo[3,4-d]benz[b]-1,4-diazin-1-one;
7-Nitro-1,2,4-oxadiazolo[3,4d]benz[b]-1,4-diazin-1-one;
5-Amino-1-(3-biphenyl)-benzimidazole;
1-(3-Biphenyl)-benzimidazole;
[N-(2-Carboxy-5-chlorophenyl)-N′-(3-trifluoromethylphenyl)]urea;
(1S,3S,4S,5S,8R)-3-(4-Chlorophenyl)-7-azatricyclo[5.3.0.0]decan-5-oxime;
(1R,2S,3S)-2-[3-(2-Thienyl)-1,2,4-oxadiazol-5-yl]-3-(2-naphthyl)-tropane;
(1R,2S,3S)-N-Normethyl-2-(3-phenyl-1,2,4-oxadiazol-5-yl)-3-(3,4-dichlorophenyl)-tropane;
[N-(2-Carboxy-5-chlorophenyl)-N′-(4-trifluoromethylphenyl)]urea;
[N-(2-Carboxy-5-chlorophenyl)-N′-(3-nitrophenyl)]urea;
(1S,3S,4S,5S,8R)-3-(3,4-Dichlorophenyl)-7-azatricyclo[5.3.0.0]decan-5-oxime;
(1R,2S,3S)-N-Normethyl-2-(3-phenyl-1,2,4-oxadiazol-5-yl)-3-(4-chlorophenyl)-tropane;
or pharmaceutically acceptable salts thereof.

5. The use according to any one of claims 2-4, wherein the disease being characterized by excessive cell death is a neurodegenerative disorder or ischemia, such as cerebral ischemia.

6. A method of treatment, prevention or alleviation of a disease in a subject, said disease being characterized by excessive or insufficient cell death, which method comprises administering to said subject a therapeutically effective amount of a compound that modulates the association of caspase-9 to Apaf-1 or a pharmaceutically acceptable amount thereof.

7. A method of treatment, prevention or alleviation of a disease in a subject, said disease being characterized by excessive cell death, which method comprises administering to said subject a therapeutically effective amount of a compound that inhibits the association of caspase-9 to Apaf-1 or a pharmaceutically acceptable amount thereof.

8. A novel compound being
[N-(2-Hydroxy-5-methoxycarbonyl-4-nitrophenyl)-N′-(3-trifluoromethylphenyl)]urea;
N-(2-Hydroxy-5-chloropheneth-2-yl)-3-trifluoromethylaniline;
[N-(2,4-Dihydroxyphenyl)-N′-(3-trifluoromethylphenyl)]urea;
1,2,4-Oxadiazolo[3,4-d]benz[b]-1,4-diazin-1-one;
1-[3-(2-Amino-pyrid-5-yl)-phen-1-yl]-5-trifluoromethyl-benzimidazoie;
5-Formamidyl-1-(3-biphenyl)-benzimidazole;
1-(3-Aminophenyl-3-phen-1-yl)-2-trifluoromethyl-benzimidazole;
1-(3-Biphenyl)-5-methoxy-benzimidazole;
[N-(2-Hydroxy-5-methoxy-4-nitrophenyl)-N′-(3-trifluoromethylphenyl)]urea;
γ-(5-Chloro-2-hydroxyphenyl)-(3-trifluoromethylacetanilide),
N-(3-Trifluoromethyl-pheneth-2-yl)-5-chloro-2-hydroxy-aniline;
[N-(5-Carboxy-2-hydroxy-4-nitrophenyl)-N′-(3-trifluoromethylphenyl)]urea;
(1R,2S,3S)-2-(3-Phenyl-1,2,4-oxadiazol-5-yl)-3-(2-naphthyl)-tropane;
[N-(2-Hydroxy-5-trifluoromethylphenyl)-N′-(3-trifluoromethylphenyl)]urea;
N,N-Bis-(2-hydroxy-5-trifluoromethylphenyl)-urea;
7,8-Dichloro-1,2,4-oxadiazolo[3,4-d]benz[b]-1,4-diazin-1-one;
7-Nitro-1,2,4-oxadiazolo[3,4-d]benz[b]-1,4-diazin-1-one;
5-Amino-1-(3-biphenyl)-benzimidazole;
1-(3-Biphenyl)-benzimidazole;
(1R,2S,3S)-2-[3-(2-Thienyl)-1,2,4-oxadiazol-5-yl]-3-(2-naphthyl)-tropane;
(1R,2S,3S)-N-Normethyl-2-(3-phenyl-1,2,4-oxadiazol-5-yl)-3-(3,4-dichlorophenyl)-tropane;
(1R,2S,3S)-N-Normethyl-2-(3-phenyl-1,2,4-oxadiazol-5-yl)-3-(4-chlorophenyl)-tropane;
or pharmaceutically acceptable salts thereof.

9. A pharmaceutical composition containing a therapeutically effective amount of a compound according to claim 8, or a pharmaceutically acceptable addition salt thereof, together with at least one pharmaceutically acceptable carrier, excipient or diluent.
